(19) United States
(12) Patent Application Publication
(10) Pub. No.: US 2004/0180868 A1
(43) Pub. Date: Sep. 16, 2004

(54) Composition and method for treating inflammations by reducing C-reactive protein

(76) Inventor: John P. Mullally, Ft. Gratiot, MI (US)

Correspondence Address: Arnold S. Weintraub, The Weintraub Group, P.L.C., Suite 240, 32000 Northwestern Highway, Farmington Hills, MI, 48334, US

(21) Appl. No.: 10/798,117

(22) Filed: Mar. 11, 2004

Related U.S. Application Data
(60) Provisional application No. 60/482,574, filed on Jun. 24, 2003.
(60) Provisional application No. 60/453,917, filed on Mar. 12, 2003.

(51) Int. Cl. A61K 31/573; A61K 31/47

(52) U.S. Cl. 514/171; 514/311

(57) ABSTRACT

A method and composition for reducing C-reactive protein for reducing systemic inflammations in the body of a user is achieved through the daily administration of a leukotriene inhibitor, and antihistamine and a corticosteroid. The composition may be administered singly or as a single medicament. Typically, the leukotriene inhibitor and antihistamine are administered orally.

### CROSS-REFERENCE TO RELATED APPLICATIONS

This application is a completion application of copending U.S. Provisional Applicaton Serial No. 60/482,574, filed Jun. 24, 2003 and 60/453,917, filed Mar. 12, 2003, the disclosures of which are hereby incorporated by reference.

### BACKGROUND OF THE INVENTION

1. Field of the Invention

The present invention pertains to medical treatments. More particularly, the present invention pertains to compositions and methods for treating certain medical conditions. Even more particularly, the present invention pertains to compositions and methods of use therefor particularly adapted for the treatment of inflammation of nasal passages, sinus cavities, and other body pathways as well as body organs.

2. Prior Art

As is known to those skilled in the art to which the present invention pertains, there have been devised many compositions for the treatment of various medical conditions. Typically, these are administered either orally or nasally. This is particularly true with both pulmonary and cavitational inflammations such as in the latter case for treating the sinuses and inflammations in the nasal passageways.

Thus, there has been developed over the years medicaments specifically adapted for utilization for treating and/or inhibiting or preventing these common and identified types of inflammations by neutralizing the source of inflammation.

For example, it is believed that leukotrienes, i.e. a group of hormone derived from arachidonic acid, mediate the allergic response that causes lung constriction and muscle contraction in asthma sufferers. To alleviate “asthma” attacks both oral liquid (inhalers) and capsules to open up the air passages and relax the muscles have been administered to the sufferer.

In practicing the present invention, it is preferred that the protocol comprise all three of the medicaments. However, it is essential that the leukotriene inhibitor be used either alone or in combination with the other components. It is to be further noted that the present protocol can be used alone or in combination with other prescription drugs which are ordinarily administered for alleviating and/or overcoming many of the diseases described hereinabove.

Similarly, histamines are potent pharmacological agents that are formed by the decarboxylation of histidine and act through receptors in smooth muscle and in secretory systems. These are stored in mast cells, are released by antigens, and are believed to be responsible for the early symptoms of anaphylaxis. In order to inhibit and prevent the dramatic effects of histamines, there have been developed a class of drugs known as antihistamines. Antihistamines are drugs that combat the histamine release during an allergic reaction by blocking the action of the histamine on the tissue. They do not stop the allergic reaction, but protect tissues from some of its effects.

Finally, there have been developed a class of anti-inflammatory intranasal sprays, which are, essentially, steroids, i.e. a generic name for lipids that contain a hydrogenated cyclopentanoperhydrophenanthrene ring system. Lipids, of course, are organic molecules which usually denote fats and terpenes. They are soluble inorganic solvents. In drug administration, usually the steroids are founded in corticosteroids, which are synthetic hormones, which include, for example, prednisone prednisolone, etc. They are anti-inflammatory and anti-allergic compounds.

However, none of the present protocols denoted above are directed to reducing C-reactive protein to minimize inflammations by using a combination of medicaments. As detailed below, it is believed that the minimization of C-reactive protein in the system alleviates a multitude of illnesses typically associated with inflammation in the body.

### SUMMARY OF THE INVENTION

The present invention provides, in a first aspect, a method for reducing and/or eliminating highly sensitive C-reactive protein in the body to eliminate inflammations, etc. by administering a composition selected from the group consisting of: (a) a leukotriene inhibitor, (b) an antihistamine, (c) a corticosteroid, and (d) mixtures thereof.

In a second aspect hereof there is provided a composition for reducing C-reactive protein which comprises an admixture of a leukotriene inhibitor, an antihistamine and a corticosteroid.

The composition is administered on a daily basis as a preventative treatment.

For a more complete understanding of the present invention reference is made to the following detailed description and accompanying non-limitative examples.

### DESCRIPTION OF THE PREFERRED EMBODIMENTS

As hereinabove noted, the present invention comprises, in a first aspect, a method for reducing and/or eliminating C-reactive protein in the body to eliminate inflammations, etc. by administering a composition selected from the group consisting of: (a) a leukotriene inhibitor, (b) an antihistamine, (c) a corticosteroid, and (d) mixtures thereof. In a second aspect hereof there is provided a composition for reducing C-reactive protein which comprises an admixture of at least to of: (a) a leukotriene inhibitor, (b) an antihistamine, and (c) a corticosteroid.

In accordance with the present invention it has been found that a daily treatment of the above-noted composition is effective in reducing highly sensitive C-reactive protein and the concomitant inhibiting of asthmatic and allergic reactions as well as other disorders as discussed hereinafter.

Generally, the leukotriene inhibitor is administered in a dosage from about 1 to 20 milligrams on a daily basis and, preferably, from about 5 to about 15 milligrams. The leukotriene inhibitor may be ingested as a pill, capsule, as a liquid, etc.

Similarly, the antihistamine is ingested, orally or nasally, on a daily basis and in an amount ranging from about 150 to 250 milligrams and, preferably, from about 175 to about 200 milligrams daily.

The corticosteroid is usually found in a liquid transport or delivery medium, such as a nasal spray or the like and ordinarily, a minimal amount ranging from about 110 μcg to about 220 μg as obtained from about 1 to about 4 nasal sprays is effective.

In using this treatment, it is preferred where all three medicaments are used that the leukotriene inhibitor and the antihistamine be administered as a pill or gelcap while the steroid, as noted, is infused as a nasal spray.

Typical of the leukotriene inhibitors are those which are selected from the group consisting of albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and the like, as well as mixtures thereof.

The inflammation reducing leukotriene inhibitor may be any of those commercially available leukotriene inhibitors such as “Zyflo®” (zileuton), “Accolate®” (zafirlukast), and “Singulair®” (a montelukast sodium) each sold commercially and available in pill form. Preferably, the leukotriene inhibitor is the montelukast sodium, which is sold commercially in pill form under the trademark “SINGULAIR®.”

The antihistamine can be any of those which are commercially available such as those sold under the name “Zyrtec®” (cetirizine), “Allegra®” (fexofenadine), “Claritin®” (loratadine), and Clarinex®.

The nasal steroid is, preferably, fluticasone propionate. This propionate is sold commercially under the name “Flonaseg.” Other useful nasal steroids are those sold commercially under various trademarks such as, for example, “Nasonex®” (mometasone furoate monohydrate), “Nasacort AQ®” (triamcinolone acetoniode), “Rhinocort Aqua®” (budesonide), and “Astelin®” (azelastine), to name a few.

According to the invention, a preferred dosage or “composition” to prevent systemic inflammation includes at least two of and, preferably, all three of a capsule of Singulair®, one capsule of Allegra®, and a prescribed squeeze of Nasonex® into each nasal passage. By taking the daily dosages, it is possible to prevent a systemic inflammation caused by the inflammatory pathways in the sinuses.

Although not wishing to be bound any theory, it is believed that the inflammation of the sinuses and nasal pathways is transmitted systemically through a (bacteria, virus, fungus) or an immune response associated with sinus drainage that is, then, transmitted through the bloodstream. It is believed that this theory also applies to other pathways including arteries, veins, and to some degree, body organs. What the present invention contemplates is the blocking of the inflamed pathways and, thus, treating this condition.

It is contemplated that the medicaments defined herein be administered at one time. It is also contemplated and within the scope hereof, that a single compound, as a liquid vehicle, be administered with the requisite amounts. In other words, there would be provided as a composition a sprayable compound having the leukotriene inhibitor, the antihistamine, and the corticosteroid all suspended in a liquid vehicle or non-toxic delivery system which can then be administered either orally or nasally through a spray and transmitted either through the mouth or the nasal passages.

Alternatively, it is possible to provide a capsule, pill or gelcap at least two or, preferably, containing all of the three components hereof.

It should further be noted that allergies cause leukotrienes to be active and, therefore, adversely affect other parts and functions of the body. It is theorized that the present method and composition provides benefits beyond the treatment of nasal inflammation. Attention is directed to Table 1 and the results obtained on several patients, each having various situations in need of medication, ranging from sleep loss, snoring, fatigue, sinus and breathing problems, hypertension, cholesterol, etc.

As noted hereinabove, the present method or protocol which for purposes of simplification is referred to hereinafter as the NAS Protocol is believed to be effective in reducing high reactive C-reactive protein (CRP). CRP is an acute phase reactant released by the body in response to acute injury, infection, or other inflammatory stimuli. As can be seen by reference to Table 1, the NAS protocol has resulted in an observable lowering of the CRP level for each of the patients, for which data is available. Similarly, it is believed that by using the present protocol while reducing overall cholesterol levels, it raises the HDL level while reducing LDL levels.

In essence, the present invention reduces or seeks to reduce CRP by minimizing and/or eliminating sinus drainage and any bacteria, virus(es), fungi or immune response associated therewith. By precluding the transmission of sinus drainage and the concomitant bacteria, etc., through the bloodstream, the pathways throughout the body including arteries, veins and body organs are prevented from becoming inflamed thereby eliminating the diseases associated therewith. By treating both pulmonary and cavitational inflammations in this manner the source of inflammation, i.e. the bacteria from the sinus drainage is eliminated.

In addition to reducing CRP and cholesterol levels it is believed that the present invention should, also, be effective in reducing and/or eliminating diabetes (Type 1 and 2), ORP, frequent infections (otitismedia, sinusitis, pneumonia, pharyngitis), asthma, chronic obstructive pulmonary disease (COPD), inflammatory lung disease (sarcoidosis), hypertension, hypercholesterolemia, renal disease, attention deficit disorder, anxiety, depression, obesity/fluctuating weight levels, irritable bowel syndrome, cardiac disease (arrhythmias, coronary artery disease, carotid stenosis, peripheral vascular disease, cerebral vascular disease, strokes), Cancer—all types, periodontal disease, sleep disorders (nightmares, sleep apnea, poor REM sleep, sleepwalking, parasomnias), migraines, radiculopathy, osteoporosis, arthritis and various other diseases attributable to systemic inflammations.

In attempting to appreciate the benefits of the present invention, it is worthy to note that bacteria, fungi, and viruses all colonize in the sinus cavity. By attacking the source those diseases which are attributable to elevated high sensitive C-reactive protein levels in the blood are, necessarily, dramatically reduced and/or eliminated. Each of the above-noted diseases have high levels of C-reactive protein associated therewith.

As to coronary or heart conditions, typically a CRP level of less than 0.5 is acceptable while a CRP level of 0.5 to 1.1 is moderate and greater than 1.1 is high risk.

High levels of CRP may help detect heart disease and coronary calcium. People with elevated CRP seem to have or develop more coronary calcium. As such, coronary calcium may be a predictor of the amount of atherosclerosis in coronary arteries, or a fatty build up in the arteries. It is believed that by following the NAS protocol herein that carotid artery blockage and stenosis associated therewith is reduced.

As to cholesterol levels, the target ranges are as follows: LDL: 50-130 mg/dL, and HDL: greater than 35 mg/dL. Depending on the Cholesterol/HDL ratio, an individual is placed into one of three risk groups: low risk: about 2.3 to 3.6; moderate risk: about 3.7 to 5.6; and high risk: about 5.7 to 10.0.

In all cases studied herein, the NAS protocol raised the “good” HDL cholesterol level while tending to lower or otherwise reduce the “bad” LDL cholesterol level. As shown in Table 1, as a result of the NAS protocol, six patients had a Cholesterol/HDL ratio of between 2.05 and 3.08 and were reduced to a “moderate risk” category, and four Patients had a Cholesterol/HDL ratio of between 3.97 and 4.63 and were reduced to a “moderate risk category”. Importantly, Patients #6 (Julie) and #12 (Ethel) went from the “high risk” category to the “moderate risk” category. In particular, for patient #6, the Cholesterol/HDL ratio went from 6.3 to 4.3 (a 28.3% reduction) and CRP was reduced by 41.9%. For Patient #12, the Cholesterol/HDL ratio was lowered from 6.6 to 4.63 (a 29.84% reduction).

In any event, though, it is the administration of these three types of compounds, which achieves the benefits of the present invention, i.e. the treatment and/or prevention of systemic inflammation, which affects many systemic diseases.

Prior to application of the protocol herein, Patients complained of an inability to sleep, snoring, fatigue and not much energy, such as from sleep loss, difficulty in breathing, such as resulting from their asthma. After following the protocol, whether for a short period measured in as little as two days, but usually, in about at least four weeks, or longer and measured in months, the patients reported an improvement in each of the aforementioned conditions. In particular, breathing was easier, and eyes were dry. Proximity of allergy triggering conditions (i.e., a smoker, a smoke filled room, an animal, etc) did not result in an inflammation, even though the nose was in some respects more sensitive.

Following the present protocol, the hemoglobin HgAlc values were 7.2 and 7.5, respectively, for two of the patients. As such, as to diabetics, blood sugar was more normal, obviating the need in some cases for reliance on diabetes medicaments. Importantly herein, the protocol reduced the HgAlc value for one of the Patients by 20.2%, from a high of 9.4. Preferably, the American Diabetes Association recommends that the hemoglobin Alc value should be less than 7%, and that treatment be pursued for patients when the value is consistently above 8%.

It is further theorized that the present method may also overcome the systemic inflammation that precludes pancreatic cells from producing insulin, thereby, potentially overcoming what is identified as Type I diabetes, as noted hereinabove.

As is known to those skilled in the art, typically a Type I diabetes sufferer is administered an antibiotic-antifungal pharmaceutical combination to threat a chronic infection. The present method can be used in conjunction with these antibiotics and antifungals for treating chronic infections.

Creatinine levels provide information as to how well the kidneys are working. Generally, the body should have a creatinine level of about between 0.5 and 1.4 mg/dL. Levels higher than 2-4 mg/dL indicate the possible presence of impairment of renal function, with a level of 10 mg/dL indicating that the kidneys are not working properly.

The present method is believed useful in lowering blood pressure in patients with diabetes and persons with renal insufficiency. Further, the composition, when administered in accordance herewith, is believed beneficial in lowering the creatinine level.

In one study, a patient was treated by the present method for about 4 months, during which time the creatinine level was reduced from 1.2 to 0.9 mg/dL. Additionally, LDL was 121.8 mg/dL (within the target range), HDL was 43.3 mg/dL (within the target range), and the Cholesterol/HDL ratio was 4.85 (moderate risk).

Further, in combination with the above noted physiological results, including control of hypertension, cholesterol, renal disorder prediction, and a lowering of CRP and/or creatinine, the present method is believed to extend to and be beneficial to weight loss; parathyroid disease, Parkinson's and Alzheimer's disease as well as prostate disease which are inflammation indicated.

In yet another aspect of the present method, the protocol is believed of benefit to sufferers of irritated bowel syndrome (“IBS”). IBS is a disorder that is found more often in women than in men, and interferes with the normal functions of the colon. The symptoms of IBS are crampy abdominal pain, bloating, constipation, and diarrhea. Although no cure has been found for IBS, the problems associated therewith can be alleviated by various treatments, including diet, stress management, and medications. Several women afflicted with IBS were placed on and benefited by the present method.

In one case, a patient (Mary, I, age 57) was treated using Accolate®, Allegra®, and Nasacort® in the above defined dosages on a daily basis for 6 to 12 months, in a “no-smoke” environment. At the end of the treatment period and following a 12-hour period of fasting, the patient exhibited lowered values of “bad cholesterol” LDL, lowered overall cholesterol, and a lowered level of triglycerides, and a raised value of the HDL cholesterol. The patient also reported better breathing and increased energy. While practicing the present invention, the patient was weaned off the use of Bentyl® (dicyclomine HCl), an anticholinergics/antispasmodic that is used to relieve cramps or spasms of the stomach, intestines, and bladder. At the end of the treatment period, the patient reported feeling better in that the pains and discomfort associated with IBS had improved.

In another case, a patient (Cora, age 62) was treated for 3 months during which time the patient typically smoked 10 cigarettes/day. At the end of the treatment period, the patient reported that the pains associated with IBS had improved. In particular, the patient reported that colon and stomach pains were reduced, abdominal pain was much reduced, and the adverse effects of cramping had gone down.

While the reason(s) why the adverse effects of IBS were lowered is not known or immediately explainable, a possible theory is that the present composition somehow controls the colonic muscle spasms and/or slows the movement of food through the digestive system.

TABLE 1
Examples Illustrating Results Using NAS Protocol
Patient Parameters: HgA1c: hemoglobin, %, and final value (in parentheses);
Chol.: Cholesterol, mg/dL; Tri.: Triglycerides, mg/dL; LDL, mg/dL; HDL, mg/dL;
Ratio of Cholesterol/HDL and final value of ratio (in parentheses); CRP: c-reactive
protein, mg/dL. The Parameters report a % change in the parameter,
the + and − indicate whether the change was an
increase/decrease, and NA indicates that data is “not available”.

| | | | | | | | Chol. | |
|---|---|---|---|---|---|---|---|---|
| | PATIENT RESULTS | HgA1c | Chol. | Tri. | LDL | HDL | HDL | CRP |
| 1 | Phoebe, Age 60. Fasted | NA | +6.3 | +46. | −8.9 | +15.3 | −9.2 | NA |
| | first, then 6 months on | | | | | | (2.05) | |
| | NAS protocol during | | | | | | | |
| | which no fasting. | | | | | | | |
| 2 | Hazel, Age 50, Fasted | NA | +17.1 | +37.7 | +6.6 | +51.1 | −22.5 | −10. |
| | first and then 6 months | | | | | | (4.42) | |
| | on NAS protocol, during | | | | | | | |
| | which no fasting. | | | | | | | |
| 3 | Patricia, Age 65, Smoke | NA | +6.7 | −11.6 | −12.9 | +69.1 | −36.6 | −10.0 |
| | environment, 11 month | | | | | | (2.66) | |
| | program, NAS protocol | | | | | | | |
| | lasted 6 months and used | | | | | | | |
| | Allegra, Singulair, and | | | | | | | |
| | Flonase. Result: Patient | | | | | | | |
| | reported feeling great, | | | | | | | |
| | and shortness of breath | | | | | | | |
| | and ability to sleep | | | | | | | |
| | improved. | | | | | | | |
| 4 | Daniel, Age 56, NAS | NA | +31.4 | +5.9 | +22.7 | +101 | −34.9 | −9.7 |
| | protocol lasted 6 weeks | | | | | | (3.97) | |
| | and used Astelin. | | | | | | | |
| | Result: Patient reported | | | | | | | |
| | feeling great, snoring, | | | | | | | |
| | congestion, and | | | | | | | |
| | nocturnal awakening | | | | | | | |
| | reduced, and energy and | | | | | | | |
| | activity improved. | | | | | | | |
| 5 | Thomas, Age 71, Fasted | NA | −6.7 | +23.9 | −17.0 | −1.4 | −6.6 | −75.0 |
| | first, then 11 months on | | | | | | (3.08) | |
| | NAS protocol, during | | | | | | | |
| | which no fasting. | | | | | | | |
| 6 | Julie, Age 42, NAS | NA | NA | NA | −5.18 | +48.9 | −28.3 | −41.9. |
| | protocol intermittent, | | | | | | (4.52) | |
| | lasted 6-8 months, | | | | | | | |
| | during which there was | | | | | | | |
| | no fasting. | | | | | | | |
| 7 | John, Age 46, NAS | NA | NA | NA | NA | NA | NA | −89.6 |
| | protocol was one month. | | | | | | | |
| | Result: Patient reported | | | | | | | |
| | sleeping better, more | | | | | | | |
| | rested in morning, and | | | | | | | |
| | nocturnal awakening | | | | | | | |
| | reduced, despite 15 | | | | | | | |
| | cigarettes/day. | | | | | | | |
| 8 | Barbara, Age 65, NAS | NA | NA | NA | NA | NA | NA | −36.0 |
| | protocol was 2 months. | | | | | | | |
| | Result: Patient reports | | | | | | | |
| | breathing improved, no | | | | | | | |
| | shortness of breath, | | | | | | | |
| | snoring reduced, and less | | | | | | | |
| | fatigued. | | | | | | | |
| 9 | Mary, Age 65, NAS | NA | NA | NA | NA | NA | NA | −75.0 |
| | protocol was 2 months | | | | | | | (1.4) |
| 10 | Marsha, Age 55, an | NA | −4.4 | −53.6 | −6.15 | +46.5 | −34.0 | NA |
| | initial NAS protocol was | | | | | | (2.97) | |
| | 2 months, a second NAS | | | | | | | |
| | protocol was 6 months. | | | | | | | |
| | Results: As to | | | | | | | |
| | hypertension, Patient | | | | | | | |
| | was weaned off Aceon, 8 mg. | | | | | | | |
| | To 4 mg, and | | | | | | | |
| | Hyzaar was stopped | | | | | | | |
| 11 | Lewis, Age 74, Patient | NA | NA | NA | NA | NA | NA | −73.3 |
| | taken off Hyzaar | | | | | | | (1.2) |
| | (hypertension stable), | | | | | | | |
| | taken off Starlix | | | | | | | |
| | (diabetes, sugars stable), | | | | | | | |
| | and decreased Lasix, 160 mg. | | | | | | | |
| | to 40 mg. | | | | | | | |
| 12 | Ethel, Age 78, NAS | 2.8 | +13.7 | −34.7 | +32.3 | +58.6 | −29.8 | NA |
| | protocol intermittent for | (7.2) | | | | | (4.63) | |
| | 6 months, not fasting | | | | | | | |
| | during protocol. | | | | | | | |
| | Weaned off Prandin | | | | | | | |
| | (diabetes). | | | | | | | |
| 13 | Judith, Age 58. As a | NA | NA | NA | NA | NA | NA | NA |
| | result of NAS protocol, | | | | | | | |
| | Patient stopped using | | | | | | | |
| | Starlix and Glocovance | | | | | | | |
| | (for diabetes) and | | | | | | | |
| | stopped using Zocor (for | | | | | | | |
| | hypercholesterol). | | | | | | | |
| 14 | David, Age 67, NAS | −20.2 | −33.1 | −67.1 | −33.4 | +7.7 | −38.0 | NA |
| | protocol was 3½ | (7.5) | | | | | (2.25) | |
| | months. | | | | | | | |
| 15 | Timothy, Age 52, on | NA | NA | NA | NA | NA | NA | −34.6 |
| | NAS protocol 6 weeks. | | | | | | | (4.9) |
| | Results: beneficial for | | | | | | | |
| | diabetes condition, | | | | | | | |
| | HgA1C lowered and | | | | | | | |
| | CRP down, and taken off | | | | | | | |
| | Avandia during NAS | | | | | | | |
| | protocol. | | | | | | | |
| 16 | William, Age 70, NAS | −20.5 | +44.34 | −43.0 | +59.7 | +56.5 | −8.0 | NA |
| | protocol beneficial for | (5.4) | | | | | (2.3) | |
| | diabetes condition. | | | | | | | |
| 17 | Edward, Age 17, a Type | 8.9 | 169 | 98 | 101 | 48 | NA | 0.29 |
| | I diabetic. After 5 weeks | | | | | | | |
| | of treatment showed | | | | | | | |
| | evidence of producing | | | | | | | |
| | his own insulin; went | | | | | | | |
| | from, 0.002 to 14 Iu/ml | | | | | | | |
| | without wearing an | | | | | | | |
| | insulin pump. | | | | | | | |

In practicing the present invention, depending on the type of condition of the patient to be treated and the severity, ordinarily improvements can be seen in anywhere from two days to about one month on.

Because of the nature of the medicaments, i.e. all over-the-counter compositions, they can be administered to most persons safely and, typically, without interference with other medicines which may be taken until the full effects of the present invention are realized.

From the above it is to be readily appreciated that there has been described herein a medical composition and method of use therefore which prevents the inflammation of the sinus cavities, while at the same time, precluding certain pulmonary activity.

1. A method for treating systemic inflammation by reducing highly sensitive C-reactive protein levels in the body of a user which comprises:
administering on a daily basis for a period of at least about 2 days, a composition selected from the group consisting of:
(a) a leukotriene inhibitor,
(b) an antihistamine,
(c) a corticosteroid, and
(d) mixtures thereof.

2. The method of claim 1 wherein the selected composition is used in an amount of:
(a) from about 1 to about 20 milligrams of leukotriene inhibitor,
(b) from about 150 to about 250 milligrams of antihistamine, and
(c) from about 110 μcg to about 220 μcg of corticosteroid.

3. The method of claim 2 wherein the selected composition is used in an amount of:
(a) from about 5 to about 15 milligrams of the leukotriene inhibitor,
(b) from about 175 to about 200 milligrams of the antihistamine, and
(c) from about 110 μcg to about 220 μcg of the corticosteroid.

4. The method of claim 2 wherein the leukotriene inhibitor is selected from the group consisting of:
albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zahamivir, and mixtures thereof.

5. The method of claim 2 wherein the antihistamine is selected from the group consisting of:
cetirizine, fexofenadine and lortadine.

6. The method of claim 2 wherein the antihistamine is selected from the group consisting of:
mometasone furoate mononhydrate, triamcinalone, acetoniode, budesonide and azelastine.

7. The method of claim 2 wherein:
(a) the leukotriene inhibitor is montelukast sodium,
(b) the antihistamine is cetirizine, fexofenadine and loratadine, and
(c) the steroid is fluticosone propionate.

8. The method of claim ______ wherein the composition comprises:
(a) the leukotriene inhibitor,
(b) the antihistamine, and
(c) the corticosteroid.

9. The method of claim 2 wherein:
the leukotriene and the antihistamine are administered orally and the steroid is nasally infused.

10. A composition for reducing C-reactive protein to treat systemic inflammation, consisting essentially of:
(a) a leukotriene inhibitor,
(b) an antihistamine, and
(c) a corticosteroid.

11. The composition of claim 10 wherein the composition comprises:
(d) from about 1 to about 20 milligrams of the leukotriene inhibitor,
(e) from about 150 to about 250 milligrams of antihistamine, and
(f) from about 110 μcg to about 220 μcg of corticosteroid.

12. The composition of claim 11 wherein the composition comprises:
(d) from about 5 to about 15 milligrams of leukotriene inhibitor,
(e) from about 175 to about 200 milligrams of antihistamine, and
(f) from about 110 μcg to about 220 μcg of corticosteroid.

13. The composition of claim 11 wherein the leukotriene inhibitor is selected from the group consisting of:
albuterol sulfate, aminophylline, amoxicillin, ampicillin, astemizole, attenuated tubercle bacillus, azithromycin, bacampicillin, beclomethasone dipropionate, budesonide, bupropion hydrochloride, cefaclor, cefadroxil, cefixime, cefprozil, cefuroxime axetil, cephalexin, ciprofloxacin hydrochloride, clarithromycin, clindamycin, cloxacillin, doxycycline, erythromycin, ethambutol, fenoterol hydrobromide, fluconazole, flunisolide, fluticasone propionate, formoterol fumarate, gatifloxacin, influenza virus vaccine, ipratropium bromide, isoniazid, isoproterenol hydrochloride, itraconazole, ketoconazole, ketotifen, levofloxacin, minocycline, montelukast sodium, moxifloxacin, nedocromil sodium, nicotine, nystatin, ofloxacin, orciprenaline, oseltamivir, oseltamivir sulfate, oxtriphylline, penicillin, pirbuterol acetate, pivampicillin, pneumococcal conjugate vaccine, pneumococcal polysaccharide vaccine, prednisone, pyrazinamide, rifampin, salbutamol, salmeterol xinafoate, sodium cromoglycate (cromolyn sodium), terbutaline sulfate, terfenadine, theophylline, triamcinolone acetonide, zafirlukast, zanamivir, and mixtures thereof.

14. The method of claim 2 wherein the antihistamine is selected from the group consisting of:
cetirizine, fexofenadine and lortadine.

15. The composition of claim 11 wherein the antihistamine is selected from the group consisting of:
(a) mometasone furoate mononhydrate,
(b) triamcinalone,
(c) acetoniode,
(d) budesonide, and
(e) azelastine.

16. The composition of claim 11 wherein:
(a) the leukotriene is montelukast sodium,
(b) the antihistamine is cetirizine, fexofenadine and loratadine, and
(c) the steroid is fluticosone propionate.

17. The composition of claim 16 wherein:
the leukotriene and the antihistamine are administered orally and the steroid is nasally infused.

18. A method for reducing sinus inflammation by reducing highly sensitive C-reactive protein to reduce systemic inflammations in the body of a user, comprising:
Administering on a daily basis a composition selected from the group consisting of:
(a) a leukotriene inhibitor,
(b) an antihistamine,
(c) a corticosteroid, and
(d) mixtures thereof.

19. The method of claim 18 wherein:
the composition is a mixture of the inhibitor, the antihistamine, and the corticosteroid.

20. The method of claim 19 wherein:
(a) the leukotriene inhibitor is a montelukast sodium present in an amount ranging from about 5 to about 15 milligrams,
(b) the antihistamine is selected from the group consisting of cetirizene, fexofenadine and lortadine present in an amount ranging from about 175 to about 200 milligrams, and
(c) the steroid is selected from the group consisting of:
momeasone furoate monohydrate, triamcinalone, acetoniode, budesonide, and azelastine, the steroid being present in an amount ranging from about 110 μcg to about 220 μcg.
